(19) United States
(12) Patent Application Publication
(10) Pub. No.: US 2025/0302568 A1
(43) Pub. Date: Oct. 2, 2025

(54) TRANSPORT DEVICE AND USE OF SAME FOR TRANSPORTING AT LEAST ONE STERILE-PRODUCT CONTAINER

(71) Applicant: Aesculap AG, Tuttlingen (DE)

(72) Inventors: Arne Jansen-Troy, Stockach (DE); Allan Maas, Konstanz (DE); Dennis Goerz, Tuttlingen (DE); Stephan Dorau, Herzogenaurach (DE); Andreas Elisch, Dunningen (DE); Stefan Thomas, Tuttlingen (DE); Matthias Schweizer, Tuttlingen (DE); Daniel Kiessling, Villingen-Schwenningen (DE); Matthias Henke, Villingen-Schwenningen (DE)

(21) Appl. No.: 18/864,358

(22) PCT Filed: May 10, 2023
(86) PCT No.: PCT/EP2023/062498

§ 371 Date: Nov. 8, 2024

(30) Foreign Application Priority Data
May 10, 2022 (DE) 10 2022 111 697.2

(51) Int. Cl. A61B 50/13; A47B 31/00

(52) U.S. Cl. CPC A61B 50/13; A47B 31/00; A47B 2031/003

(57) ABSTRACT

A transport device for transporting a sterile-product container a includes a trolley and support elements on a first level and on a second level. A receptacle is storable on the support elements. A lifting support is arrangeable in a first position such that the receptacle is movable back and forth between the support elements of the first level and the lifting support. The lifting support is arrangeable in a second position such that the receptacle is movable back and forth between the support elements of the second level and the lifting support. The transport device also includes a guide extending transversely to the first and second levels. The guide guides the lifting support from the first position into the second position. The transport device further includes a drive, by which the lifting support is movable along the guide from the first position into the second position.

### CROSS-REFERENCE TO RELATED APPLICATIONS

This application is the United States national stage entry of International Application No. PCT/EP2023/062498, filed on May 10, 2023, and claims priority to German Application No. 10 2022 111 697.2, filed on May 10, 2022. The contents of International Application No. PCT/EP2023/062498 and German Application No. 10 2022 111 697.2 are incorporated by reference herein in their entireties.

### FIELD

The present invention relates to a transport device for transporting at least one sterile-product container having a receiving space which has support elements on a first plane and on a second plane parallel to the first plane, wherein a sterile-product container receptacle, configured in particular as a tray, is supportable on the support elements, and having a lifting support which is arrangeable in a first position on the support elements of the first plane in such a way that the sterile-product container receptacle is displaceable back and forth between the support elements of the first plane and the lifting support.

### BACKGROUND

EP 2 594 453 B1 discloses a transport device for transporting at least one sterile-product container having a receiving space which has support elements on a first plane and on a second plane parallel to the first plane, wherein a sterile-product container receptacle is supportable on support elements. The known transport device has a lifting support or a lift device which are arrangeable on the support elements of the first plane in such a way that the sterile-product container receptacle is displaceable back and forth between the support elements of the first plane and the lifting support.

The problem with the transport device according to EP 2 594 453 B1 is that its lifting support or lift device is only designed for support elements of a single plane. The known lift device only serves to be able to insert a small sterile-product container receptacle in the form of a slide-in rack into a transport trolley, which is actually configured for transporting a larger sterile-product container receptacle in the form of a slide-in rack.

### SUMMARY

The object of the present disclosure is therefore to provide a transport device for transporting at least one sterile-product container, which enables space-saving transportation of several sterile-product containers in an ergonomic manner.

This object is solved by a transport device and its use for transporting at least one sterile-product container.

A transport device according to the disclosure for transporting at least one sterile-product container has a receiving space and a lifting support.

The receiving space is configured in a movable transport trolley and has support elements on a first plane and on a second plane parallel to the first plane, wherein a sterile-product container receptacle is supportable on the support elements. The support elements may in particular be projections projecting from sidewalls of the receiving space, which have surfaces or contact surfaces facing upward which can come into contact with the sterile-product container receptacle or with surfaces or contact surfaces of the sterile-product container receptacle facing downward, in particular flat surfaces. The sterile-product container receptacle may be configured in particular as a tray. Alternatively, the sterile-product container receptacle may also be configured as a slide-in rack.

The lifting support is configured to be arranged or arrangeable in a first position on the support elements of the first plane in such a way that the sterile-product container receptacle is displaceable back and forth between the support elements of the first plane and the lifting support. In particular, the lifting support may be configured to be arranged or arrangeable in the first position on the support elements of the first plane in such a way that the lifting support extends parallel to the support elements of the first plane or to the contact surfaces of the support elements of the first plane and in particular is aligned therewith.

Furthermore, the lifting support is configured to be arranged or arrangeable in a second position on the support elements of the second plane in such a way that the sterile-product container receptacle is displaceable back and forth between the support elements of the second plane and the lifting support. In particular, the lifting support may be configured to be arranged or arrangeable in the second position on the support elements of the second plane in such a way that the lifting support extends parallel to the support elements of the second plane or to the contact surfaces of the support elements of the second plane and in particular is aligned therewith.

The transport device has a linear guide, in particular in the form of at least one guide rail, which extends transversely, in particular perpendicularly, to the first and second planes and via which the lifting support can be guided from its first position to its second position. In addition, the transport device has a drive, in particular a linear drive, preferably in the form of an electromechanical actuator, integrated in the linear guide or the guide rail, via which the lifting support is movable along the linear guide from the first position to the second position.

The linear guide, the drive, and the lifting support are configured on the transport trolley.

The transport device according to the disclosure advantageously enables the receiving space to be filled more comprehensively with sterile-product containers without excessive physical strain.

According to an aspect of the disclosure, the transport trolley may have a frame surrounding an opening through which the sterile-product container may be moved into or out of the receiving space, the transport trolley may have a door pivotably mounted on an outer front side of the frame, and the linear guide, the drive as well as the lifting support may be configured on an inner side of the door in such a way that the lifting support may be aligned parallel to the support elements of the receiving space in an open position of the door.

If the lifting support is configured separately from the door on the inner side of the door, the lifting support can be protected from external influences in an advantageous way.

According to one aspect of the disclosure, the lifting support may be pivotably connected to the linear guide in such a way that the lifting support can be folded from a folded-out position, in which the lifting support is aligned parallel to the support elements of the receiving space in the open position of the door, into a folded-in position, in which the lifting support or its contact surface, which is designed or configured for contact with the sterile-product container receptacle, is aligned parallel to the door or its inner side.

If the lifting support is configured to be pivotable with the linear guide, the dimensions of the lifting support can be advantageously adapted to the size of the sterile-product container receptacle or to the sterile-product container to be transported.

According to an aspect of the disclosure, the door may be configured as a French door having a first door leaf and a second door leaf, and the lifting support may comprise a first lifting-support element configured on the first door leaf and a second lifting-support element configured on the second door leaf. A face plate is formed in particular by a central portion of the frame. In other words, the door may be configured as a double-wing swing door, the two door wings of which are pivotably connected to the frame in such a way that pivot axes of the two door wings, in particular extending perpendicular to the first plane, are arranged on two opposite sides of the frame.

If the door is configured as a French door or double-wing swing door, the load that occurs between the first and second plane when the sterile-product container is displaced can be distributed better.

According to an aspect of the disclosure, the transport trolley may have a frame surrounding an opening through which the sterile-product container may be moved into or out of the receiving space, and the linear guide, the drive, and the lifting support may be configured on an outer front side of the frame.

If the linear guide, the drive, and the lifting support are configured on or integrally with the transport trolley, ergonomic loading and unloading can be achieved regardless of location.

According to one aspect of the disclosure, the lifting support may be configured as a door which is pivotably and displaceably connected to the linear guide in such a way that the door or lifting support can at least largely, in particular completely, cover the opening surrounded by the frame in a covering orientation, and the door or lifting support is aligned parallel to the support elements or its contact surfaces of the receiving space in a lifting orientation aligned transversely, in particular perpendicularly, to the covering orientation, in which in particular the opening is partially, in particular largely, not covered by the door or lifting support.

If the lifting support is configured as a door, the ergonomic loading and unloading of the transport trolley can be realized efficiently.

According to an aspect of the disclosure, the door may be configured as a folding door whose door leaves are aligned in the covering orientation in an unfolded state and in the lifting orientation in a folded state. In particular, the linear guide may have two guide rails arranged on opposite sides of the front side of the frame. In particular, the drive per guide rail may have an actuator for displacing the door leaves.

If the lifting support is configured as a folding door, the dimensions of the lifting support may advantageously be adapted to the size of the sterile-product container receptacle or of the sterile-product container to be transported.

According to an aspect of the disclosure, the drive per door leaf may have at least one actuator for displacing the respective door leaf along the linear guide. In particular, the linear guide may comprise two guide rails arranged on opposite sides of the front side of the frame. In particular, the drive may have one actuator for displacing the respective door leaf for each door leaf and for each guide rail. In particular, the actuators of one of the two guide rails may be actuated synchronously with the actuators of the other one of the two guide rails.

If at least one actuator is provided for each door leaf, it is advantageously possible to automatically displace the lifting support relative to the first and second plane and also to unfold and/or fold the folding door.

According to one aspect of the disclosure, at least one bracing element movable relative to the door may be provided on the door, wherein the bracing element is movable into a bracing position, in particular linearly, in which the bracing element can support the door in its open position relative to a flat base on which the transport trolley is supported. In particular, the transport trolley may have at least one bracing element per door wing. Preferably, at least one bracing element may be provided at the free end of each door wing.

If the transport trolley has at least one bracing element, the probability of the transport trolley toppling over when the door is open can be advantageously reduced.

The present disclosure further relates to use of a transport device according to the disclosure for transporting at least one sterile-product container.

### BRIEF DESCRIPTION OF THE DRAWING FIGURES

The present invention will be described in more detail below with reference to the accompanying drawings via preferred configuration examples. The following is shown:

FIGS. 1 to 4 show perspective views of an embodiment of a transport device not covered by the present invention in various positions of a lifting support or of a sterile-product container receptacle;

FIGS. 5 to 14 show perspective views of a first embodiment of a transport device according to the disclosure in different positions of a lifting support or of a sterile-product container receptacle; and

FIGS. 15 to 22 show perspective views of a second embodiment of a transport device according to the disclosure in different positions of a lifting support or of a sterile-product container receptacle;

### DETAILED DESCRIPTION

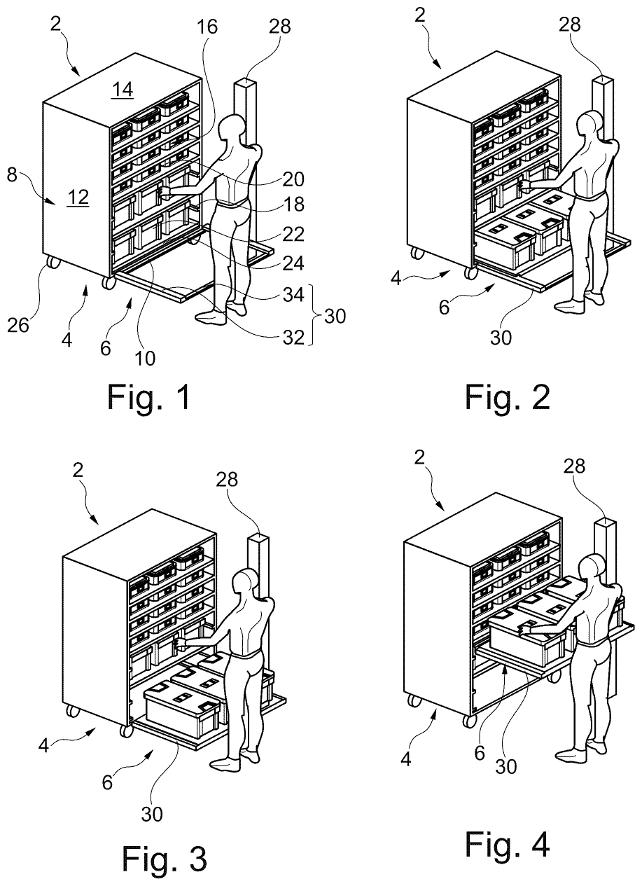

FIG. 1 shows a perspective view of a transport device 2 not covered by the present invention with a transport trolley 4 and a stationary lifting device 6 separate from the transport trolley.

The transport trolley 2 is configured as a roll-fronted cabinet and has a housing 8 with a bottom 10, two opposing sidewalls 12 extending upward from the bottom 10 and a cover plate 14 opposite the bottom 10. The housing 8 or the bottom 10, the sidewalls 12 and the cover plate 14 frame a receiving space 16. Support elements 18 are configured on the inner sides or on sides of the sidewalls 12 facing the receiving space 16, which project into the receiving space 16. The support elements 18 are preferably configured as strips. The support elements 18 are arranged in such a way that two opposing support elements 18 each lie on a common plane.

In order to be able to receive or store sterile-product containers 20 or 22, the transport trolley 4 has sterile-product container receptacles 24 in the form of trays. The sterile-product container receptacles 24 are dimensioned in such a way that they can each be placed on two opposing support elements 18 and in this state extend in particular parallel to the bottom 10. The sterile-product container receptacles 24 have geometries such as depressions and/or elevations (see FIG. 14) in order to ensure defined placement of the sterile-product containers 20 or 22 and to prevent or at least make it more difficult for the sterile-product containers 20 or 22 to slip during transportation.

The sterile-product container receptacles 24 may be placed in the transport trolley 2 or on support elements 18 at a distance from each other in such a way that there is enough space for large sterile-product containers 22 between individual (preferably lower) sterile-product container receptacles 24 or between one of the sterile-product container receptacles 24 and the bottom 10/the cover plate 14, and that between other sterile-product container receptacles 24 or between one of the sterile-product container receptacles 24 and the bottom 10/the cover plate 14 there is only enough space for small sterile-product containers 20.

An upper side of the bottom 10 is configured in particular in such a way that it also forms a support element 18 or has support elements 18.

The support elements 18 may be configured with geometries (not shown) in order to ensure a defined placement of the sterile-product container receptacles 24 and to prevent or at least make it more difficult for the sterile-product container receptacles 24 to slip during transportation. These geometries on the support elements 18 may be configured to be movable in particular in order to secure a sterile-product container receptacle 24 in a secured position and to allow displacement of the sterile-product container receptacle 24 in an open position.

In order to enable the transport trolley 4 to be moved, rollers 26 are provided on a lower side of the bottom 10, in each case near a corner of the bottom 10, which are configured in particular as swivel castors.

The lifting device 6 has a linear guide 28 with an integrated drive (not shown), in particular in the form of a linear drive, and a lifting support 30 displaceably connected to the linear guide 28.

The linear guide 26 is firmly connected to a base and extends substantially vertically.

The lifting support 30 has two opposing lifting-support elements 32, which are connected to each other via connecting crosspieces 34. The lifting-support elements 32 and the connecting crosspieces 34 together form a rectangular frame. The lifting-support elements 32 are raised on an upper side of the lifting support 30 relative to the connecting crosspieces 34 in such a way that the lifting-support elements 32 can form rails for the sterile-product container receptacles 24.

In the state shown in FIG. 1, the lifting support 30 is in a lowermost position or orientation in which the lifting support 30 is arranged on the upper side of the bottom 10 or on the support elements 18 of the bottom 10 in such a way that the corresponding lowermost sterile-product container receptacle 24 is displaceable back and forth between the upper side of the bottom 10 or the support elements 18 of the bottom 10 on the one hand and the lifting support 30 or the two lifting-support elements 32 on the other hand.

In the state shown in FIG. 2, the lowest sterile-product container receptacle 24, together with the large sterile-product containers 22 stored on it, is being pushed from the receiving space 16 onto the lifting support 30.

In the state shown in FIG. 3, the lowest sterile-product container receptacle 24, together with the large sterile-product containers 22 stored on it, is pushed completely onto the lifting support 30.

Via the linear guide 28 and the drive integrated therein, the lifting support 30 can be lifted from the position shown in FIGS. 1 to 3. For example, the lifting support can be raised such that it is arranged in a second position on the support elements 18 of a plane spaced from the bottom 10 such that the previously lowermost sterile-product container receptacle 24 is displaceable back and forth between the support elements of the plane spaced from the bottom 10 and the lifting support 30. In the states shown in FIGS. 3 and 4, the transport trolley 4 is fully loaded except for the lowest plane, so that a sterile-product container receptacle 24 would first have to be removed for a change of position of sterile-product container receptacles 24.

In the state shown in FIG. 4, the lifting support 30, together with the previously lowest sterile-product container receptacle 24 and the large sterile-product containers 22 stored on it, is raised to a position in which a person can remove one of the large sterile-product containers 22 from the sterile-product container receptacle 24 in an ergonomic manner.

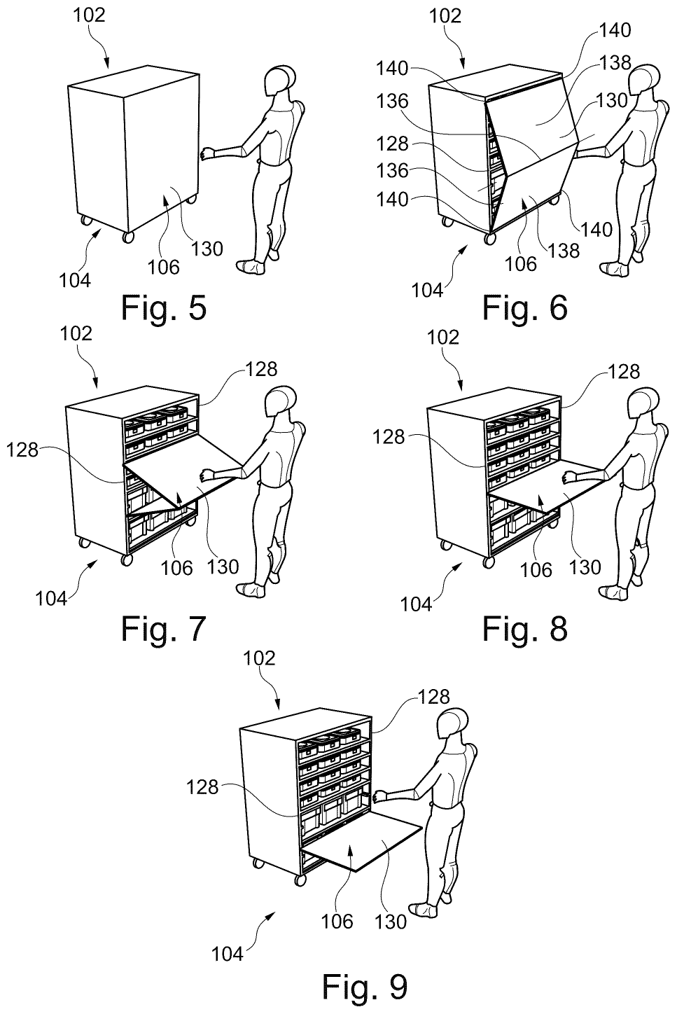

FIGS. 5 to 14 show perspective views of a transport device 102 according to the disclosure according to a first embodiment with a transport trolley 104.

The transport trolley 104 is configured as a roll-fronted cabinet and has a housing 108 with a bottom 110, two opposing sidewalls 112 extending upward from the bottom 110 and a cover plate 114 opposite the bottom 110.

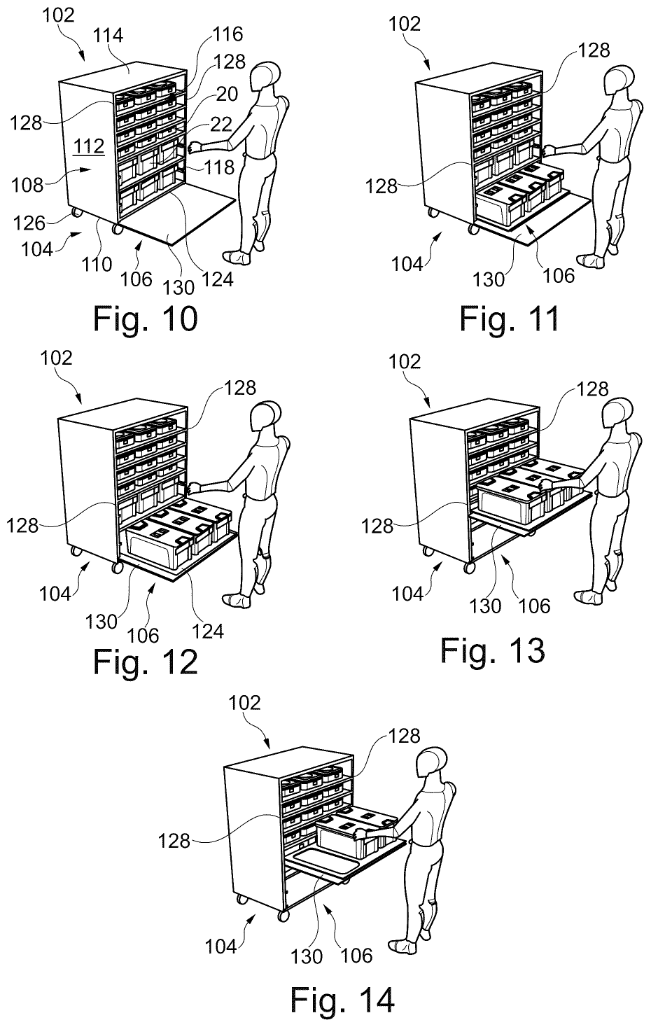

As shown in FIG. 10, the bottom 110, the sidewalls 112, the cover plate 114, the receiving space 116, support elements 118, sterile-product container receptacles 124 as well as rollers 126 of the transport trolley 104 according to the first embodiment are configured according to the bottom 10, the sidewalls 12, the cover plate 14, the receiving space 16, the support elements 18, the sterile-product container receptacles 24 and the rollers 26 of the transport trolley 2 according to the embodiment not covered by the present invention.

In contrast to the transport device 2 according to the embodiment not covered by the present invention, the transport device 102 according to the first embodiment comprises a lifting device 106 configured on the transport trolley 104.

The lifting device 106 has a lifting support 130 configured as a folding door and in a closed state the lifting support 130 covers an opening framed by the bottom 110, the sidewalls 112 and the cover plate 114. For this purpose, a vertically extending guide rail is configured on each of the front sides of the sidewalls 112, which together form a linear guide 124 for the plate-shaped lifting support 130.

As shown in FIG. 6, the lifting support 130 has two door leaves 138 hingedly connected to each other about a folding axis 136. The lifting support 130 is configured in such a way that the folding axis 136 of the door leaves 138 is aligned parallel to the bottom 110. The door leaves 138 are each pivotably and displaceably connected to both guide rails of the linear guide 128 by an end opposite the folding axis 136 at corresponding contact points 140. In order to be displaced relative to the linear guide 128, the guide rails have a drive (not shown) or in each case two actuators (not shown) at the contact points 140.

In the state shown in FIG. 5, the door leaves 138 of the lifting support 128 are in an unfolded state, in which the door leaves 138 are aligned vertically and cover the receiving space 116.

In the state shown in FIG. 8, the door leaves 138 of the lifting support 128 are in a folded state, in which the door leaves 138 are aligned horizontally, so that sterile-product containers 20 and/or 24 can be placed on the door leaves 138 or one of the sterile-product container receptacles 124 with corresponding sterile-product containers 20 and/or 24 can be placed on the door leaves 138.

FIGS. 6 and 7 show intermediate states during folding of the door leaves 138.

The folded door leaves 138 can be lowered and raised in translation along the guide rails of the linear guide 128, for example starting from the state shown in FIG. 8.

FIGS. 9 and 10 show how the folded door leaves 138 are displaced onto a lowermost plane, so that the lifting support 130 formed by the folded door leaves 138 is arranged on an upper side of the bottom 110 or on support elements 118 of the bottom 110 in such a way that the corresponding lowermost sterile-product container receptacle 124 is displaceable back and forth between the upper side of the bottom 110 or the support elements 118 of the bottom 110 on the one hand and the lifting support 130 on the other hand.

FIGS. 11 to 13 show states corresponding to FIGS. 2 to 4.

In the state shown in FIG. 11, the lowest sterile-product container receptacle 124, together with the large sterile-product containers 22 stored on it, is pushed from the receiving space 16 onto the lifting support 30.

In the state shown in FIG. 12, the lowest sterile-product container receptacle 124, together with the large sterile-product containers 22 stored on it, is pushed completely onto the lifting support 130.

Via the linear guide 128 and the drive integrated therein or the actuators integrated therein, the lifting support 130 can be lifted from the position shown in FIGS. 10 to 12. For example, the lifting support 130 can be lifted such that it is arranged in a second position against the support elements 118 of a plane spaced from the bottom 110 such that the previously lowermost sterile-product container receptacle 124 is displaceable back and forth between the support elements of the plane spaced from the bottom 110 and the lifting support 130. In the states shown in FIGS. 12 and 13, the transport trolley 104 is fully loaded except for the lowermost plane, so that a sterile-product container receptacle 124 would first have to be removed for a change of position of sterile-product container receptacles 124.

In the state shown in FIG. 13, the lifting support 30, together with the previously lowest sterile-product container receptacle 124 and the large sterile-product containers 22 stored on it, is raised to a position in which a person can remove one of the large sterile-product containers 22 from the sterile-product container receptacle 24 in an ergonomic manner.

FIG. 14 shows a state in which a sterile-product container 22 is unloaded from the sterile-product container receptacle 124 stored on the lifting support 130.

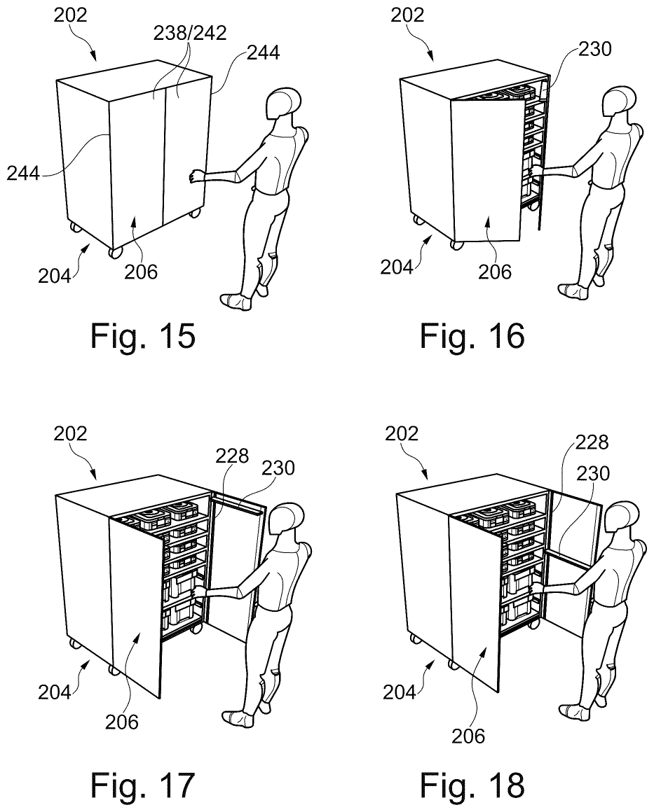

FIGS. 15 to 22 show perspective views of a transport device 202 according to the disclosure according to a second embodiment with a transport trolley 204.

The transport trolley 204 is configured as a roll-fronted cabinet and has a housing 208 with a bottom 210, two opposing sidewalls 212 extending upward from the bottom 210 and a cover plate 214 opposite the bottom 210.

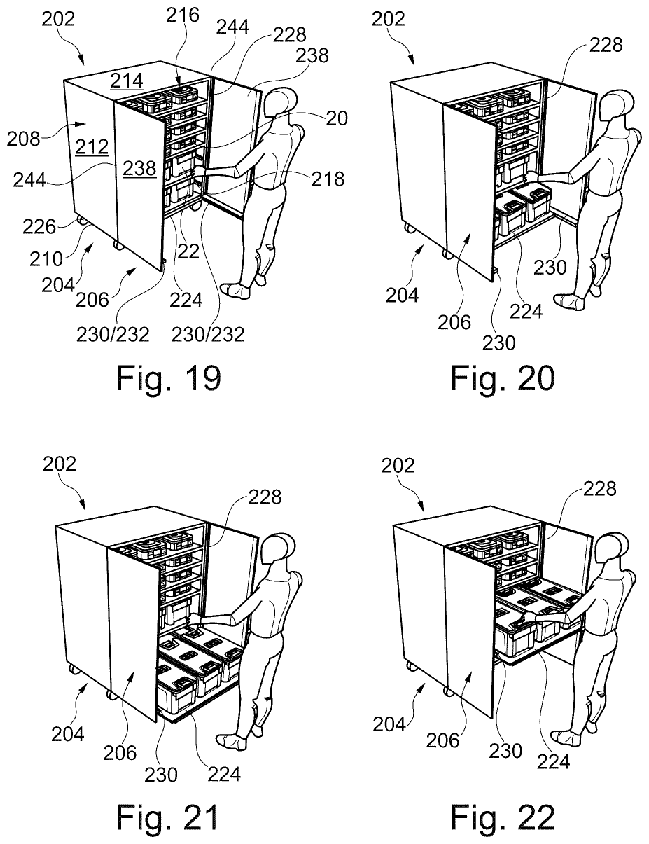

As shown in FIG. 19, the bottom 210, the sidewalls 212, the cover plate 214, the receiving space 216, support elements 218, sterile-product container receptacles 224 as well as rollers 226 of the transport trolley 204 according to the second embodiment are configured according to the bottom 10, the sidewalls 12, the cover plate 14, the receiving space 16, the support elements 18, the sterile-product container receptacles 24 as well as the rollers 26 of the transport trolley 2 according to the embodiment not covered by the present invention.

In contrast to the transport device 2 according to the embodiment not covered by the present invention, the transport device 202 according to the second embodiment, like the transport device 102 according to the first embodiment, has a lifting device 206 configured on the transport trolley 204.

The lifting device 206 has a double-wing swing door 242 with two door leaves 238. The two door leaves 238 are each pivotably attached to a front side of the corresponding sidewall 212 via vertically extending hinges 244 in such a way that the door leaves 238 in a closed position (see FIG. 15) cover an opening framed by the bottom 210, the sidewalls 212 and the cover plate 214 and in an open position (see for example FIG. 17) each extend the corresponding sidewall 212 or are each aligned in the same (vertical) plane as the directly adjacent sidewall 212.

Both door leaves 238 each have a lifting-support element 232 and two guide rails serving as linear guides 228. The respective lifting-support element 232 is arranged on an inner side of the corresponding door leaf 238, which faces the receiving space 216 when the door leaf 238 or the door leaves 238 are in the closed position, parallel to the bottom 210 or parallel to the cover plate 214 or parallel to the planes defined by the support elements 218. The respective guide rails are arranged at two opposite edges of the corresponding door leaf 238 transversely or perpendicularly to the bottom 210 or to the cover plate 214 or to the planes defined by the support elements 218. The lifting-support elements 232 are configured as strips, the two ends of which are pivotably and displaceably connected to one of the two guide rails of a respective door leaf 238. The lifting-support elements 232 can be displaced along the corresponding guide rails and can be pivoted or folded relative to the corresponding door leaf 238 into an unfolded position and into a folded-in position. In the folded-in position, the lifting-support elements 232 abut the corresponding door leaf 238 such that the lifting-support elements 232 do not or only partially protrude into an area in front of the receiving space 216 when the door leaves 238 are in the open position. In the folded-out position, the lifting-support elements 232 project away from the corresponding door leaf 238 in such a way that a sterile-product container receptacle 224 can be placed on the two lifting-support elements 232 when the door leaves 238 are in the open position and the two lifting-support elements 232 are at a common level.

An actuator is provided in at least one of the two guide rails of each door leaf 238, preferably in both guide rails, via which the corresponding lifting-support element can be at least displaced, preferably displaced and pivoted. The two lifting-support elements 232 together form the lifting support 230.

In the state shown in FIG. 15, the door leaves 238 are in the closed position.

In the state shown in FIG. 17, the door leaves 238 are in the open position and the lifting-support elements 238 or lifting support 230 are at the level of a lower side of the cover plate 214.

FIG. 16 shows a state during the opening of the swing door 242 or the door leaves 238.

FIGS. 18 and 19 show how the lifting support 230 is displaced to a lowermost plane, so that the lifting support 230 formed by lifting-support elements 232 is arranged on an upper side of the bottom 210 or on support elements 218 of the bottom 210 in such a way that the corresponding lowermost sterile-product container receptacle 224 is displaceable back and forth between the upper side of the bottom 210 or the support elements 218 of the bottom 210 on the one hand and the lifting support 230 on the other hand.

FIGS. 20 to 22 show states corresponding to FIGS. 2 to 4.

In the state shown in FIG. 20, the lowest sterile-product container receptacle 224, together with the large sterile-product containers 22 stored on it, is pushed from the receiving space 216 onto the lifting support 230.

In the state shown in FIG. 21, the lowest sterile-product container receptacle 224, together with the large sterile-product containers 22 stored on it, is pushed completely onto the lifting support 230.

Via the linear guide 228 and the drive integrated therein or the actuators integrated therein, the lifting support 230 can be lifted from the position shown in FIGS. 19 to 21. For example, the lifting support 230 can be lifted such that it is arranged in a second position against the support elements 218 of a plane spaced from the bottom 210 such that the previously lowermost sterile-product container receptacle 224 is displaceable back and forth between the support elements of the plane spaced from the bottom 210 and the lifting support 230. In the states shown in FIGS. 21 and 22, the transport trolley 204 is fully loaded except for the lowest plane, so that a sterile-product container receptacle 224 would first have to be removed for a change of position of sterile-product container receptacles 224.

In the state shown in FIG. 22, the lifting support 230 together with the previously lowermost sterile-product container receptacle 224 and the large sterile-product containers 22 stored thereon is raised to a position in which a person can remove one of the large sterile-product containers 22 from the sterile-product container receptacle 24 in an ergonomic manner.

The lifting supports 30; 130 or 230 according to the disclosure or the corresponding drives can be controlled in particular via operating elements (not shown). In particular, various positions of the lifting supports are stored in the operating elements or in corresponding control units, which correspond to the planes defined by the support elements 18; 118 or 218. Preferably, the operating elements for the lifting supports 103 and 230 are arranged on one of the door leaves 138 or 238. The sterile-product container receptacles 124 and 224 are configured in particular in such a way that they can interact in a form-fitting manner with the corresponding linear guide 128 and 228, so that the sterile-product container receptacles 124 and 224 cannot be detached from the transport device 102 and 202, or at least not without further action.

The transport trollies 4; 104 or 204 according to the disclosure may be configured in particular in such a way that the sterile-product container receptacles 24; 124 or 224 can be pushed along the support elements 18; 118 or 218 from and/or into the receiving space 16; 116 or 216 via drives provided for this purpose.

The transport trollies 4; 104 or 204 according to the disclosure may be configured as driverless transport systems (DTS) or automated guided vehicles (AGV).

The transport trollies 104 or 204 according to the disclosure may each be provided with a mechatronic lock, via which the respective door can be locked in the closed position. The respective lock may preferably be configured such that the lock is released when the corresponding transport trolley 104 or 204 is at predetermined positions or a predetermined position.

### List of Reference Signs

2; 102; 202 transport device
4; 104, 204 transport trolley
6; 106; 206 lifting device
8; 108; 208 housing
10; 110; 210 bottom
12; 112; 212 sidewall
14; 114; 214 cover plate
16; 116; 216 receiving space
18; 118; 218 support element
20, 22 sterile-product container
24; 124; 224 sterile-product container receptacle
26; 126; 226 scooter
28; 128, 228 linear guide
30; 130; 230 lifting support
32; 232 lifting-support element
34 connecting crosspiece
136 folding axis
138; 238 door leaf
140 contact point
242 swing door
244 hinge

1.-10. (canceled)

11. A transport device for transporting at least one sterile-product container, the transport device comprising:
a receiving space configured in a transport trolley;
a lifting support;
a linear guide; and
a drive,
the receiving space comprising support elements on a first plane and on a second plane parallel to the first plane, the support elements configured such that a sterile-product container receptacle is supportable on the support elements,
the lifting support being arrangeable in a first position on the support elements of the first plane in such a way that the sterile-product container receptacle is displaceable back and forth between the support elements of the first plane and the lifting support,
the lifting support also being arrangeable in a second position on the support elements of the second plane in such a way that the sterile-product container receptacle is displaceable back and forth between the support elements of the second plane and the lifting support,
the linear guide extending transversely to the first plane and the second plane, such that lifting support is guidable from the first position to the second position by the linear guide,
the lifting support being movable along the linear guide from the first position to the second position by the drive, and
the linear guide, the drive, and the lifting support being configured on the transport trolley.

12. The transport device according to claim 11, wherein:
the transport trolley has a frame surrounding an opening through which the at least one sterile-product container is moveable into or out of the receiving space,
the transport trolley has a door pivotably mounted on an outer front side of the frame, and
the linear guide, the drive, and the lifting support are configured on an inner side of the door in such a way that the lifting support is alignable parallel to the support elements in an open position of the door.

13. The transport device according to claim 12, further comprising at least one bracing element movable on the door that is moveable relative to the door, wherein the at least one bracing element is movable into a bracing position in which the at least one bracing element supports the door in the open position relative to a flat base on which the transport trolley is supported.

14. The transport device according to claim 12, wherein the lifting support is pivotably connected to the linear guide in such a way that the lifting support is foldable from a folded-out position, in which the lifting support is aligned parallel to the support elements of the receiving space in the open position of the door, into a folded-in position, in which the lifting support is aligned parallel to the door.

15. The transport device according to claim 14, further comprising at least one bracing element movable on the door that is moveable relative to the door, wherein the at least one bracing element is movable into a bracing position in which the at least one bracing element supports the door in the open position relative to a flat base on which the transport trolley is supported.

16. The transport device according to claim 12, wherein:
the door is configured as a French door having a first door leaf and a second door leaf, and
the lifting support comprises a first lifting-support element configured on the first door leaf and a second lifting-support element configured on the second door leaf.

17. The transport device according to claim 16, further comprising at least one bracing element movable on the door that is moveable relative to the door, wherein the at least one bracing element is movable into a bracing position in which the at least one bracing element supports the door in the open position relative to a flat base on which the transport trolley is supported.

18. The transport device according to claim 11, wherein:
the transport trolley has a frame surrounding an opening through which the at least one sterile-product container is movable into or out of the receiving space, and
the linear guide, the drive, and the lifting support are configured on an outer front side of the frame.

19. The transport device according to claim 18, wherein:
the lifting support is configured as a door which is pivotably and displaceably connected to the linear guide in such a way that the door at least largely covers the opening in a covering orientation, and
the door is aligned parallel to the support elements of the receiving space in a lifting orientation aligned transversely to the covering orientation.

20. The transport device according to claim 19, wherein the door is configured as a folding door comprising door leaves that are aligned in the covering orientation in an unfolded state and in the lifting orientation in a folded state.

21. The transport device according to claim 20, wherein the drive comprises at least one actuator for displacing the door leaves along the linear guide.

22. The transport device according to claim 21, further comprising at least one bracing element movable on the door that is moveable relative to the door, wherein the at least one bracing element is movable into a bracing position in which the at least one bracing element supports the door in an open position relative to a flat base on which the transport trolley is supported.

23. The transport device according to claim 20, further comprising at least one bracing element movable on the door that is moveable relative to the door, wherein the at least one bracing element is movable into a bracing position in which the at least one bracing element supports the door in an open position relative to a flat base on which the transport trolley is supported.
